(19) United States
(12) Patent Application Publication
(10) Pub. No.: US 2007/0243247 A1
(43) Pub. Date: Oct. 18, 2007

(54) Dosage Form Containing The Active Ingredient Cholylsarcosine

(76) Inventors: Jennifer Dressman, Frankfurt (DE); Hans-Jürgen Stein, Homburg (DE); Thomas Furst, Frankfurt (DE); Christiane Bott, Langenselbold (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/569,581

(22) PCT Filed: May 12, 2005
(86) PCT No.: PCT/EP05/05157

§ 371 Date: Nov. 24, 2006

(30) Foreign Application Priority Data
Jun. 9, 2004 (DE) 10 2004 027 924.1

(51) Int. Cl. A61K 9/16; A61K 9/20

(52) U.S. Cl. 424/464; 424/490

(57) ABSTRACT

The invention relates to a dosage form, containing the active ingredient cholylsarcosine, in the form of pellets, which are provided with a polymer coating that is resistant to gastric juices. The invention is characterised in that it discloses pellets comprising an active ingredient, which contain between 50 and 80 wt. % of the active ingredient cholylsarcosine and between 50 and 20 wt. % of one or more conventional pharmaceutical adjuvants as binding agents, whereby at least 90 wt. % of said adjuvants are water-soluble and the size of at least 80 % of the pellets comprising an active ingredient is between 800 and 2,500 $g(m)m. The granulates containing an active ingredient are coated with an anonic, film-forming polymer coating agent, which dissolves in a 0.07M sodium phosphate buffer with a pH value of 5.5 at a dissolution rate of at least 10 mg/min*g and whose dissolution rate in a 0.07M sodium phosphate buffer with a pH value of 6.0 is at least 200 mg/min*g. The polymer coating amounts to between 5 and 15 wt. % of the pellet weight. The invention also relates to a method for producing said dosage form.

The invention relates to a dosage form comprising the active ingredient cholylsarcosine and to a process for its production.

### Prior Art

In patients with short bowel syndrome it is possible owing to an impaired reabsorption of bile salts for the pool of bile acids to be diminished. The result is an intestinal malabsorption of fats and fat-soluble food constituents. Cholylsarcosine, a semisynthetic bile salt, is suitable for oral replacement therapy but may cause gastrointestinal irritation.

Meyer J. H., Elashoff J., Porter-Fink V., Dressmann J. and Amidon G. L. describe in Gastroenterology 1988, 94, 1315-1325 under the title “Human Postprandial Gastric Emptying of 1-3-Millimeter Spheres” pancreatin-containing microspheres which are proposed for the therapy of pancreatic insufficiency. Microspheres with a size range of 1.4+/−0.3 mm are particularly suitable in this connection for passing simultaneously with the chyme from the stomach into the bowel.

U.S. Pat. No. 4,976,949 (Meyer et al.) describes active ingredient-containing systems in multiparticulate form or oral intake, which are transported with the chime, with a ratio of density and diameter being described in a formula-like relationship

Fürst, The Bott C., Herbert E., Zygoura, D., Stein J. and Dressman J B describe on a poster with the title “Coated Cholylsarcosin Granulates for the Treatment of Short Bowel Syndrome”, which was shown at the Digestive Disease Week or May 19, 2003/ American Gasteroenterology Association, Orlando, a dosage form comprising the active ingredient cholylsarcosine. For this purpose, cholylsarcosine-containing pellets are produced by wet granulation and coated with a polymer which is resistant to gastric juice but which is not defined. The particle size of the coated pellets is below 1 mm,. The in vitro release profile shown on the poster shows a cholylsarcosine release at pH 45 of somewhat less than 20% after 20 min. A suitable dosage of the active ingredient may be 4 g per day.

Problem and Solution

The intention is to improve the formulation of cholylsarcosine pellets which is resistant to gastric juice and is in the size range below 1 mm as shown by Fürst et al (2003) in such a way that the dosage form can be taken together with a meal and displays a faster effect. It is intended in particular that the cholylsarcosine pellets pass with the chyme from the stomach into the bowel and rapidly release the active ingredient there.

The problem is solved by a dosage form comprising the active ingredient cholylsarcosine in the form of active ingredient-containing pellets which are provided with a polymer coating resistant to gastric juice, characterized in that the active ingredient-containing pellets employed comprise 50-80% by weight of the active ingredient cholylsarcosine and 50 to 20% by weight of one or more pharmaceutically usual excipients as binders, where at least 90% by weight of the excipients present are soluble in water, and at least 80% of the active ingredient-containing pellets have a size in the range from 800 to 2500 μm and where the active ingredient-containing pellets are coated with an anionic, film-forming polymeric coating composition which dissolves in 0.07M sodium phosphate buffer of pH 5.5 with a dissolution rate of at least 10 mg/min*g, and whose dissolution rate in 0.07M sodium phosphate buffer of pH 6.0 is at least 200 mg/min*g, where the polymeric coating accounts for 5 to 15% by weight based on the pellet weight,

and the dosage form releases not more than 10% of the contained active ingredient after 60 min at pH 1.2 and releases at least 30% of the contained active ingredient after 20 min at pH 4.5.

The invention is based on the realization that the cholylsarcosine formulation resistant to gastric juice as shown by Fürst et al. (2003) is to be improved in such a way that it releases at least 30% of the contained active ingredient after 20 min at pH 4.5 Surprisingly, this is possible as claimed without the disadvantageous effect that more than 10% of the contained active ingredient is released after 60 min at pH 1.2. Despite rapid release of active ingredient on passing from the stomach into the bowel, the gastric juice-resistant effect remains fully retained so that unwanted side effects do not occur.

Implementation of the Invention

Dosage form comprising the active ingredient cholylsarcosine in the form of active ingredient-containing pellets which are provided with a polymer coating resistant to gastric juice.

The active ingredient-containing pellets employed comprise 50 to 80, preferably 70 to 78, % by weight of the active ingredient cholylsarcosine and 50 to 20, preferably 30 to 22, % by weight of one or more pharmaceutically usual excipients as binders. Below the lower limit it is possible only with difficulty to provide the comparatively high daily dose of from 2 to 4 g of cholylsarcosine in such a way that it can be taken by the patient in a reasonable manner. A dose of, for example, 4 capsules each of 0.5 g of active ingredient twice a day would presumably just about be accepted by the patient. Intake of a larger number of units each with a smaller amount of active ingredient would probably meet with less acceptance (“patient compliancy”) and would also be more risky because of the possibility of miscounting.

The pharmaceutically usual excipients or binders which are employed are intended to bind the active ingredient and contribute to the possibility of producing attrition-resistant and maximally rounded pellets of the desired size by mixing the components and adding liquid. Pelleting or granulation processes are known to the skilled worker and described in the literature (e.g. Lieberman H E; Lachman L; Schwartz J B: Pharmaceutical Dosage Forms: Tablets Volume 1 and 3 second edition; Marcel Dekker Inc 1990).

At least 90, preferably at least 95, particularly preferably 100, % by weight of the employed pharmaceutically usual excipients or binders should be soluble in water. This favors rapid solution of the pellets after the coating film which is resistant to gastric juice has dissolved.

Under soluble in water will amount to a solubility in water of the excipients used of at least 300 g/l.

The binder preferably employed is a mixture of sucrose and polyvinylpyrrolidone (e.g. Kollidon 25) A quantitative ratio of 7 to 9 parts of sucrose to 1 to 3 parts of polyvinylpyrrolidone is favorable. For production, for example the sucrose can be mixed dry with the active ingredient, and the polyvinylpyrrolidone can be added dropwise or sprayed in as solution in water or ethanol/water in a high-speed mixer.

At least 80% of the active ingredient-containing pellets should have a size in the range from 800 to 2500, preferably 1000 to 2000, μm. This size ensures that the passage from the stomach into the bowel together with the chyme is still sufficiently fast.

A skilled worker is able to adjust the process parameters for example so that pellets with an average size approximately in the region of 1500 μm are produced. The necessary particle size fraction is obtained by subsequent grading (sieving) with the assistance of sieves having different exclusion limits.

The active ingredient-containing pellets are coated with an anionic, film-forming polymeric coating composition which dissolves in 0.07M sodium phosphate buffer of pH 5.5 with a dissolution rate of at least 10 mg/min*g ([mg/min×g]) and whose dissolution rate in 0.07M sodium phosphate buffer of pH 6.0 is at least 200 mg/min*g. The dissolution rate is determined in this connection with the aid of glass beads coated with the polymer. The glass beads are put into the phosphate buffer to be investigated, and the dissolution rate is ascertained by means of a pH-stat method. The pH of the investigation solution is kept constant by titration with 0 5M sodium hydroxide solution over a defined period, and the dissolution rate can be calculated from the consumption of sodium hydroxide solution and the linear region of the resulting titration plot (see also pamphlet Diss. Rate/E 2003/10; degussa/Röhm Pharma Polymere). The coating composition is practically insoluble in the pH range below 5.0 and therefore serves as coating resistant to gastric juice. In the transitional range from about pH 4.0 to 5.0, the polymer film swells and becomes permeable. It is thus possible for active ingredient to be released even in this pH range.

An example of a suitable film-forming coating is a methacrylate copolymer which is polymerized from 40 to 60% by weight of ethyl acrylate and 60 to 40% by weight of methyl methacrylate (Eudragit® L100-55 type).

Also suitable as film-forming coating is a hydroxy-propylmethylcellulose phthalate (HPMCP).

The polymeric coating is relatively thin and accounts for only 5 to 15, preferably 8 to 12, % by weight based on the pellet weight. This is also important in order not to administer too high a dose of the coating composition, which might cause possible side effects, with the high daily dose of active ingredient.

In order to be able to apply such a relatively thin coating uniformly, the active ingredient-containing pellets should be maximally rounded. A good rounding can be reproduced inter alia by means of the characteristics of friability, bulk density, tapped density and angle of repose.

The active ingredient-containing pellets employed should therefore preferably have a friability (attrition) of not more than 0.5, in particular not more than 0.4, %.

The friability is a measure of the resistance to attrition of the pellets or granules. A skilled worker is able to determine the friability for example with the aid of a commercial friabilizator (e.g. Erweka, Heusenstamm). A defined amount of pellets is introduced into the instrument and exposed to an attrition-causing rotation for a particular time. Suitable conditions are, for example, 2 g of product (initial weight) rotation at 20 revolutions per minute with a test time of 5 minutes. The friability is calculated from the difference in mass of the removed pellets divided by the original weight (Ph. Eur.).

The active ingredient-containing pellets employed should further preferably have a bulk density in the range from 3.5 to 0.7 g/ml.

A skilled worker is able to determine the bulk density by measuring the volume and weight of the pellets and calculating the ratio of mass and volume (g/ml) (Ph. Eur.).

The active ingredient-containing pellets employed should further preferably have a tapped density in the range from 0.6 to 0.8 g/ml.

A skilled worker is able to determine the tapped density by compacting the pellets to constant volume, e.g. with the assistance of a tapping volumimeter, and calculating the ratio of the initial weight and the final volume (g/ml) (Ph. Eur.).

The active ingredient-containing pellets employed should preferably have an angle of repose of the active ingredient-containing granules in the range below 60, in particular below 55, degrees.

A skilled worker is able to determine the angle of repose by allowing the powder to be investigated to run out of a funnel onto a flat substrate and measuring the angle of the surface of the cone consisting of the powder against the substrate. A smaller result for this angle means better flow behavior of the bulk material

The dosage form releases not more than 10, preferably not more than 5, % of the contained active ingredient after 60 min at pH 1.2, and at least 30, preferably at least 35 or at least 40, % of the contained active ingredient after 20 min at pH 4.5.

The active ingredient release is carried out in a USP XXIII apparatus 2 (paddle; 100 rpm) dissolution tester. With the release profile being determined at pH 1.2 from 500 ml of simulated gastric fluid sine pepsin (SGFsp; USP XXIII), whereas 500 ml of phosphate buffer (Ph. Eur. 2000NT) is used to investigate the release profile at pH 4.5. The release media are to be degassed and maintained at 37° C. during the test in accordance with USP XXIII. Analytical evaluation of the investigations of content and release takes place by an HPLC method.

The dosage form is preferably a multiparticulate dosage form.

Possible examples are tablets compressed from pellets, minitablets, pellets-containing capsules, sachets or reconstitutable powders. Sachets are preferred in particular because intake of high single doses is comparatively simple.

Production Processes

The dosage form can be produced by mixing 50-80, preferably 70 to 78, % by weight of the active ingredient cholylsarcosine with 50 to 20, preferably 30 to 22, % by weight of one or more pharmaceutically usual excipients as binders, where at least 90, preferably at least 95 or 100, % by weight of the excipients are soluble in water, and rounding in a manner known per se to give pellets, at least 80% of which have a size in the range from 800 to 2500 μm. The rounding can take place for example in a high-speed mixer (fast-running forced-action mixer) with the assistance of liquid.

The excipients or the binders can for example be in part premixed dry with the active ingredient, while another part of the binder is added dropwise or sprayed into water or an organic solvent or an appropriate mixture of, for example, ethanol/water. It is possible and preferred to choose at the start of the mixing and rounding process a slow speed of rotation which car be increased towards the end of the process. It is also beneficial to install blades in the mixer which counteract agglomeration. The active ingredient-containing pellets are expediently dried at the end of the rounding process so that the introduced liquid is substantially or entirely removed again.

It is possible to employ as binder for example a mixture of sucrose and polyvinylpyrrolidone (e.g. Kollidon). A quantitative ratio of 7 to 9 parts of sucrose to 1 to 3 parts of polyvinylpyrrolidone is beneficial. In the production it is possible for example for the sucrose to be mixed dry with the active ingredient and for the polyvinylpyrrolidone to be added dropwise or sprayed in as solution in water or ethanol/water (e.g. in the ratio 50-50) in a high-speed mixer.

The substantially rounded, dried, active ingredient-containing pellets are subsequently coated in a manner known per se with the anionic film-forming polymeric coating composition, the intention being to apply the polymeric coating in an amount of from 5 to 15, preferably 8 to 12, % by weight based on the weight of the pellets. The result is a dosage form which releases not more than 10, preferably not more than 5, % of the contained active ingredient after 60 min at pH 1.2 and at least 30, preferably at least 40, % of the contained active ingredient after 20 min at pH 4.5.

The pellets with a coating resistant to gastric juice can be further processed in a manner known per se to give a multiparticulate dosage form. The dosage form is suitable for the therapy of short bowel syndrome.

### EXAMPLES

Production of the Pellets—Comparative Example

A granulation liquid is prepared by dissolving the binder Kollidon VA 64 (15.5 g) in 123.5 g of water. The active ingredient (630 g) and the granulation aid Saccharose pulvris (70 g) are weighed and blended in a paddle mixer. The granulation liquid is then incorporated in portions into the powder mixture until it reaches fast ball consistency. The moist composition is then broken down using a pestle and formulation sieves of size 4 and 3 to granules of the desired particle size and dried in a tray dryer at 40° C. for 12 h.

Production of the Pellets—Example According to the Invention

The granulation liquid is prepared by dissolving 8 g of Kollidon VA 64 in 60 g of ethanol (50%) The active ingredient (300 g) and the granulation aid Saccharose pulvris (100 g) are put into the product container of a high-speed mixer (Rotolab; Zanchetta) and mixed at a rotor speed of 200 rpm for 5 min. The granulation liquid is then added dropwise to the powder mixture (addition time 7 min), during which the rotor speed is raised to 300 rpm. For rounding and for further buildup of the pellets, the rotor speed is raised to 400 rpm for 2 min and the chopper (5000 rpm) is switched on. Subsequently the resulting material is dried at a rotor speed of 120 rpm (interval of 100 sec on/600 sec off). During the drying process, the heating jacket of the apparatus is heated to 80° C. and simultaneously vacuum (25 mbar) is applied. The drying time is 30 min.

Polymeric Coating

In order to prepare the required spray suspension, the aqueous dispersion of the polymer is weighed (17 g) and the plasticizer triethyl citrate (1 g) is added and stirred with a magnetic stirrer overnight. In a second mixture, water is heated to 75° C., and glycerol monostearate GMS (0.5 g) and Tween 80 (0.1 g) are incorporated with continuous stirring (Ultra Turrax) until the completely molten GMS forms a milky homogeneous emulsion. Before starting the spraying process, the GMS emulsion is slowly added with continuous stirring to the polymer dispersion.

The uncoated pellets from the comparative example and the example according to the invention (50 g) are each put in separate batches in the product container of a Miniglatt fluidized bed apparatus, and the polymer is applied in the bottom-spray process with a Wurster insert. A spray nozzle with a diameter of 0.5 mm is used, and the spraying pressure is 0.7 bar with a spraying rate of 0.95 g/ml. The inlet air temperature of 40° C. ensures a product temperature of 28° C. The total processing time including 10 min after-drying time is 33 min. The pellets of the comparative example received an amount of coating of 20% by weight based on the weight of the pellets. The pellets of the example according to the invention received an amount of coating of 10% by weight based on the weight of the pellets.

TABLE 1

| | Comparative | Example according |
|---|---|---|
| | example*⁾ | to the invention |
| Cholylsarcosine [wt %]**⁾ | 88 | 73.1 |
| Sucrose [wt %] | 9.8 | 24.5 |
| Polyvinylpyrrolidone [wt %] | 2.2 | 2.9 |
| 80% of pellets in size range | <1 mm | 1 to 2 mm |
| Coating composition | Eudragit ® | Eudragit ® |
| | L100-55***⁾ | L100-55 |
| Amount of coating [wt %] | 20 | 10 |
| Active ingredient release | 8 | 4 |
| pH 1.2 after 60 [%] | | |
| Active ingredient release | 18 | 40 |
| pH 4.5 after 20 min [%] | | |

*⁾= The comparative experiment was carried out using unpublished data but technical correspondence to the poster publication discussed at the outset by Fürst et all. “Coated Cholylsarcosin Granulates for the Treatment of Short Bowel Syndrome”, Digestive Disease Week 2003, American Gasteroenterology Association, Orlando
**⁾[wt %] in each case based on the weight of pellets
***⁾Copolymer polymerized from 50% by weight ethyl acrylate and 50% by weight methacrylic acid

1: A dosage form comprising the active ingredient cholylsarcosine in the form of active ingredient-containing pellets that are provided with a polymer coating resistant to gastric juice,
wherein
the active ingredient-containing pellets comprise 50 to 80% by weight of the active ingredient cholylsarcosine and 50 to 20% by weight of one or more pharmaceutically usual excipients as binders, where at least 90% by weight of the excipients used are soluble in water, and at least 80% of the active ingredient-containing pellets have a size in the range from 800 to 2500 μm, and where
the active ingredient-containing pellets are coated with an anionic, film-forming polymeric coating composition that dissolves in 0.07M sodium phosphate buffer of pH 5.5 with a dissolution rate of at least 10 mg/min*g, and whose dissolution rate in 0.07M sodium phosphate buffer of pH 6.0 is at least 200 mg/min*g,
where the polymeric coating accounts for 5 to 15% by weight based on the pellet weight.

2: The dosage form as claimed in claim 1, wherein a methacrylate copolymer that polymerizes from 40 to 60% by weight of ethyl acrylate and 60 to 40% by weight of methyl methacrylate is used as film-forming coating.

3: The dosage form as claimed in claim 1, wherein a hydroxypropylmethylcellulose phthalate (HPMICP) is used as film-forming coating.

4: The dosage form as claimed in claim 1, wherein a mixture of sucrose and polyvinylpyrrolidone is used as binder.

5: The dosage form as claimed in claim 1, wherein the active ingredient-containing granules have a friability of not more than 0.5%.

6: The dosage form as claimed in claim 1, wherein the active ingredient-containing granules have a bulk density in the range from 0.5 to 0.7 g/ml.

7: The dosage form as claimed in claim 1, wherein the active ingredient-containing granules have a tapped density in the range from 0.6 to 0.8 g/ml.

8: The dosage form as claimed in claim 1, wherein the active ingredient-containing granules have an angle of repose in the range below 60 degrees.

9: The dosage form as claimed in claim 1, wherein the dosage form is a multiparticulate dosage form.

10: The dosage form as claimed in claim 9, wherein the dosage form is in the form of tablets compressed from pellets, minitablets, pellets-containing capsules, sachets or reconstitutable powders.

11: A process for producing a dosage form as claimed in claim 1, comprising mixing
50-80% by weight of the active ingredient cholylsarcosine with 50 to 20% by weight of one or more pharmaceutically usual excipients as binders, where at least 90% by weight of the contained excipients are soluble in water, and are rounded to pellets, at least 80% of which have a size in the range from 800 to 2500 μm, and
coating the active ingredient-containing pellets with an anionic, film-forming polymeric coating composition that dissolves in 0.07M sodium phosphate buffer of pH 5.5 with a dissolution rate of at least 10 mg/min*g and whose dissolution rate in 0.07M sodium phosphate buffer of pH 6.0 is at least 200 mg/min*g, where a polymeric coating is applied in an amount of from 5 to 15% by weight based on the weight of the pellets, and a dosage form which releases not more than 10% of the contained active ingredient after 60 min at pH 1.2 and at least 30% of the contained active ingredient after 20 min at pH 4.5 is obtained.

12: The process as claimed in claim 11, further comprising processing the pellets (granules) with a coating resistant to gastric juice in a manner known per se to give a multiparticulate dosage form.
